(19) United States
(12) Patent Application Publication
(10) Pub. No.: US 2013/0041040 A1
(43) Pub. Date: Feb. 14, 2013

(54) SOLID PHARMACEUTICAL COMPOSITION FOR BUCCAL ADMINISTRATION OF AGOMELATINE

(71) Applicants: François THARRAULT, Orleans (FR); Cécile POIRIER, Orleans (FR); Gilles FONKNECHTEN, Ligny le Ribault (FR); Jean-Manuel PEAN, Orleans (FR)

(72) Inventors: François THARRAULT, Orleans (FR); Cécile POIRIER, Orleans (FR); Gilles FONKNECHTEN, Ligny le Ribault (FR); Jean-Manuel PEAN, Orleans (FR)

(73) Assignee: LES LABORATOIRES SERVIER, Suresnes Cedex (FR)

(21) Appl. No.: 13/570,626

(22) Filed: Aug. 9, 2012

(30) Foreign Application Priority Data
Aug. 10, 2011 (FR) 11/02500

(51) Int. Cl. A61K 31/165; A61P 25/22; A61P 25/00; A61P 9/00; A61P 1/00; A61P 3/04; A61P 25/24; A61P 25/20

(52) U.S. Cl. 514/630

(57) ABSTRACT

The invention relates to a solid buccal pharmaceutical composition comprising agomelatine intended for systemic action.

The present invention relates to a new solid pharmaceutical form for administration of agomelatine by the buccal route.

Agomelatine, or N-[2-(7-methoxy-1-naphthyl)ethyl]acetamide of formula (I):

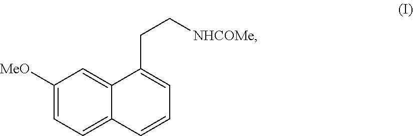

and its hydrates, crystalline forms, complexes, co-cristals, and addition salts with a pharmaceutically acceptable acid or base has valuable pharmacological properties: it is a selective agonist of receptors of the melatoninergic system and, on the other hand, an antagonist of the 5-HT_{2C} receptor. These properties provide it with activity in the central nervous system and, more especially, in the treatment of major depression, seasonal affective disorder, generalized anxiety disorder, obsessional compulsive disorder, bipolar disorders, sleep disorders, cardiovascular pathologies, pathologies of the digestive system, insomnia and fatigue due to jet-lag, appetite disorders and obesity.

Agomelatine, its crystalline forms, its complexes, its co-crystals, its addition salts with a pharmaceutically acceptable acid or base, its preparation and its use in therapeutics have been described, among others, in the patent applications EP0447285, WO2005/077887, WO2007/015003, WO2007/015002, WO2007/015004, WO2010/097052, WO2010/102554, CN101955440, WO2011/050742, CN102050756, WO2011/006387, WO2011/075943, CN101774937, CN101870662, CN102030673, WO2012/046253, WO2011/113362, WO2011/113363, CN102206864, CN102503886, and CN102432490.

In all that follows hereinbelow, “agomelatine” is understood to mean agomelatine, its hydrates, its complexes, its co-crystals, its crystalline forms and its addition salts with a pharmaceutically acceptable acid or base.

The agomelatine may especially be in crystalline form II.

Agomelatine can be administered by the oral route or, more specifically, by the enteral route in the form of immediate-release tablets to be swallowed with half a glass of water. These agomelatine tablets are useful, especially in treating major depression, seasonal affective disorder, generalized anxiety disorder, obsessional compulsive disorder, bipolar disorders, sleep disorders, cardiovascular pathologies, pathologies of the digestive system, insomnia and fatigue due to jet-lag, appetite disorders and obesity, and any pathology associated with deregulation of circadian rhythms.

Pharmacokinetic studies in humans have shown that the bioavailability of agomelatine by the oral route is low compared to the parenteral route and varies for one and the same individual and from one individual to another.

The low bioavailability of agomelatine and the variations in inter- and intra-individual concentrations have therefore resulted in the search for a new formulation allowing those drawbacks to be overcome. A solid orodispersible pharmaceutical composition of agomelatine, described in Patent Application EP1427724, was therefore developed, containing agomelatine and granules consisting of lactose and starch dried by co-atomisation and marketed under the name STARLAC®. This pharmaceutical composition makes it possible to obtain tablets having a very good capacity for disintegrating in the oral cavity and, more specifically, in the sublingual cavity, meeting the criteria for orodispersibility. The orodispersible tablets make it possible to deliver the active ingredient into the oral cavity and, more specifically, into the sublingual cavity in less than three minutes. Dissolution of the active ingredient in the saliva and then absorption via the mucous membranes of the oral cavity and, more specifically, the sublingual mucous membrane, and rapid passage into the blood make it possible to avoid presystemic degradation and the hepatic first-pass effect. Accordingly, bioavailability is very clearly improved with much lower variability and rapid appearance of the active ingredient in the blood.

However, it quickly became apparent that this formulation had a drawback due to the active ingredient used—agomelatine—which causes a pronounced sensation of irritation in the mucous membranes of the oral cavity.

When the sublingual route is specifically targeted, this stinging effect is exacerbated because of the high local concentrations of agomelatine, the end result of which is very poor patient acceptability. In this context, a formulation of the sublingual tablet type was developed and described in Patent Application WO2007/068829, meeting the requirements for orodispersibility and patient acceptability. The favoured strategy was to obtain a solid pharmaceutical composition composed of a central core or central layer containing agomelatine and excipients allowing an orodispersible formulation to be obtained, and an orodispersible coating optionally containing a counter-irritant. These orodispersible tablets make it possible to obtain rapid disintegration in less than 3 minutes and have shown an excellent ability to limit the stinging character of the active ingredient.

The Applicant has now followed a new strategy consisting of developing a pharmaceutical formulation allowing gradual release of agomelatine in the buccal cavity. The important point of this new formulation is to maintain, throughout release of the active ingredient, local concentrations which are sufficiently low to limit the stinging sensation and to obtain a form acceptable to the patient.

Accordingly, a new pharmaceutical formulation was developed, making it possible to remedy the problem of irritation by the agomelatine whilst favouring delivery of the active ingredient into the buccal cavity for the purpose of systemic action, that effect being sought in order to improve the problems of bioavailability and intra- and inter-individual variability with tablets administered by the oral route. In surprising and completely unexpected manner, this new formulation has resulted in an increase in the bioavailability of the active ingredient not only compared to the oral (enteral) tablet but also compared to the orodispersible oromucosal formulations developed in the prior art for the purpose of systemic action, for formulations having the same dosage in active principle and wherein the active principle has the same form. By “same form” it is understood, here and every time it will be used hereinafter, that the active principle has the same crystalline form, or is under the same hydrate, complexe, salt or co-crystal form, and that particle sizes of active principle are the same.

The present invention accordingly relates to a solid buccal preparation intended for systemic action, which makes possible controlled release of the active ingredient in the oral cavity so as to obtain good acceptability in terms of taste and better absolute bioavailability compared to the orodispersible tablets having an oromucosal absorption described in the prior art, for formulations having the same dosage in active principle and wherein the active principle is under the same form.

The buccal preparation according to the invention relates more especially to lozenges, pastilles to be sucked, tablets to be sucked, sublingual tablets, gingival tablets, solids obtained by hot-melt extrusion or injection-moulding or also medicament-containing spheres intended to dissolve in the oral cavity, which contain agomelatine.

The buccal preparation according to the invention may contain diluents, binders, flow agents, lubricants, coating agents, plasticisers, flavourings, counter-irritants and sweeteners.

The buccal preparation according to the invention is characterised by the absence of a disintegrant, which, combined with a high compression strength, makes it possible to provide a tablet which does not disintegrate but erodes slowly by dissolution of the constituents on the surface.

The systemic-action buccal preparation according to the invention has an active ingredient release time less than 30 minutes, and more preferably less than 15 minutes. The active ingredient release time will be for example between 3 minutes and 30 minutes, and more preferably between 3 minutes and 15 minutes.

Lozenges and pastilles to be sucked are solid unit-dose preparations intended to be sucked and to dissolve or disintegrate slowly in the mouth. Lozenges to be sucked are hard preparations obtained by moulding. They contain the active ingredient usually in a flavoured and sugary excipient. They may optionally contain counter-irritants. Pastilles to be sucked are soft and malleable preparations obtained by moulding mixtures containing gums or natural or synthetic polymers and sweeteners. They may optionally contain flavourings and counter-irritants.

Lozenges and pastilles to be sucked, in accordance with the invention, will comprise, for example, agomelatine, gum arabic, mono- or di-saccharides such as glucose or sucrose, polysaccharides, isomalt, maltodextrin or polyols such as maltitol.

Tablets to be sucked are solid unit-dose preparations intended to be sucked in order to exert a local or systemic action. They are obtained by direct compression or by granulation followed by compression.

The pharmaceutical composition according to the invention will be, for example, a tablet to be sucked which may be prepared by a direct compression process. The tablet to be sucked, in accordance with the invention, comprises agomelatine, a diluent and, optionally, a binding agent, a lubricating agent and/or a flow agent and/or one or more sweetening, flavouring or counter-irritation agents. It is characterised by slow erosion in the oral cavity. The diluent that may be used in accordance with the invention is so selected that it allows use in direct compression and provides the pharmaceutical form with high resistance to crushing and acceptable organoleptic properties for a buccal preparation to be sucked. The diluent used is preferably a polyol or a saccharide (mono-, oligo- or poly-saccharide) or a combination of those various compounds such as, for example, glucose, sucrose, mannitol, dextrates or dextrin.

Among the binding agents that may be used in accordance with the invention there may be mentioned cellulose compounds and starch compounds, crospovidone and maltodextrin. The counter-irritant optionally used is a compound acting on the vanilloid receptors or the ASIC (acid-sensitising ion channel) receptors responsible for sensations of irritation and pain. Preference is given to the counter-irritant used being citric acid.

The other excipients used are excipients customarily described for each of the categories in reference works such as, for example, the Handbook of Pharmaceutical Excipients (Rowe, Sheskey and Owen, Pharmaceutical Press).

Among the lubricating agents that may be used in accordance with the invention there may be mentioned magnesium stearate, stearic acid, glycerol behenate, sucroesters and sodium stearyl fumarate, more preferably magnesium stearate or sodium stearyl fumarate.

Among the sweeteners optionally envisaged according to the invention there may be mentioned aspartame, acesulfame potassium, sucralose and saccharin.

The flavourings optionally envisaged according to the invention may comprise any compound intended to have an effect relating to the taste descriptors (saltiness, sweetness, bitterness, sourness, umami).

Sublingual and gingival tablets are solid unit-dose preparations intended for application beneath the tongue and in the gingivo-buccal space, respectively, for the purpose of systemic action. They are produced by compression of mixtures of powders or granules so as to obtain tablets having a shape matched to their intended use.

Sublingual and gingival tablets according to the invention will comprise the same ingredients as tablets to be sucked. They have similar release profiles. They differ from tablets to be sucked only in their administration route. Tablets to be sucked are intended for administration via the oromucosal route, that is to say absorption of the active ingredient occurs via the entirety of the mucous membranes of the buccal cavity, whereas sublingual tablets are applied specifically by the sublingual route, that is to say beneath the tongue, and gingival tablets specifically by the gingival route, that is to say via the gums.

The solids obtained by hot-melt extrusion or injection-moulding are prepared using the same ingredients as tablets to be sucked, but thermoplastic polymers are specifically added in order to provide the mixture of ingredients with flow properties in the hot state. Shaping of the solid is obtained by extrusion through a die and then calendering and cooling or by injection of the material into a cooled mould. The thermoplastic polymers used in accordance with the invention are compounds customarily described in reference works such as, for example, the Polymer Handbook (Brandrup, Immergut and Grulke, Wiley-Interscience). There may be mentioned, for example, cellulose compounds, poloxyethylenes, polyvinylpyrrolidones or polymethacrylates. Plasticising compounds may optionally be used in order to facilitate flow and shaping of the material. Plasticisers used in accordance with the invention are plasticisers customarily described in reference works such as, for example, the Handbook of Pharmaceutical Excipients (Rowe, Sheskey and Owen, Pharmaceutical Press). There may be mentioned, for example, dibutyl sebacate, triacetin, glycerol, sorbitol and polyethylene glycols.

The solid buccal preparation intended for systemic action in accordance with the invention, after administration, has shown excellent active ingredient acceptability to the patient. The formulation developed in accordance with the invention does indeed allow gradual dissolution of the agomelatine, and therefore slow and controlled release, avoiding the introduction of an especially irritating very large amount of the active substance.

The Applicant has moreover carried out pharmacokinetic studies in humans and has been able to demonstrate, in entirely unexpected and surprising manner, much better bioavailability for a solid pharmaceutical composition for buccal administration according to the invention compared to the orodispersible tablets described in the prior art, for formulations having the same dosage in active principle, and wherein the active principle has the same form.

The pharmaceutical composition according to the invention has therefore made it possible to increase the bioavailability of the active ingredient very significantly and, as a consequence, to reduce intra- and inter-individual variability, whilst offering the patient a highly acceptable formulation.

The invention accordingly relates to a solid buccal pharmaceutical composition comprising agomelatine, intended for systemic action, resulting in an increase in the bioavailability of the active ingredient compared especially to an orodispersible form administered by the sublingual or oromucosal route. More especially, the pharmaceutical composition according to the invention makes it possible to attain absolute bioavailability of the active ingredient of more than 25%, and more preferably of more than 30%, corresponding to an increase by a factor of close to 1.5 to 2 compared to the sublingual or oromucosal orodispersible formulations of the prior art, when formulations having the same dosage in active principle and wherein the active principle has the same form are compared.

The pharmaceutical compositions according to the invention are preferably characterised in that they contain, in relation to the total weight of the composition, from 0.01% to 20% agomelatine by weight, more especially from 0.01% to 10%, and even more preferably from 0.01% to 5%.

The pharmaceutical compositions according to the invention preferably comprise a diluent having a very good compression capability, whilst providing the formulation with an erodable character. Among the diluents according to the invention there may be mentioned saccharides and polyols, more preferably sucrose.

The useful dosage can be varied according to the nature and severity of the disorder, the administration route and the age and weight of the patient. The dosage varies from 0.1 mg to 25 mg of agomelatine per day in one or more administrations, and preferably from 0.1 mg to 10 mg of agomelatine per day in one or more administrations, and even more preferably from 0.1 mg to 5 mg of agomelatine per day in one or more administrations.

The Examples that follow illustrate the invention but do not limit it in any way:

### EXAMPLE 1

### Formulation

### Tablet to be Sucked, Having a Weight of 500 Mg

| Constituents | Amount (mg) |
|---|---|
| Agomelatine | 2 |
| Sucrose for direct compression | 434.25 |
| Pregelatinised maize starch | 50 |
| Aspartame | 5 |
| Acesulfame potassium | 5 |
| Magnesium stearate | 3.75 |

The tablet to be sucked is prepared by mixing the constituents (with four premixing steps) followed by direct compression with a compression force of 30 kN and at a speed of 30,000 tablets per hour. The tablets obtained have a hardness of 50 N.

Examples 2 and 3 are obtained in accordance with the same process:

### EXAMPLE 2

### Formulation

### Tablet to be Sucked, Having a Weight of 500 Mg

| Constituents | Amount (mg) |
|---|---|
| Agomelatine | 0.5 |
| Sucrose for direct compression | 435.75 |
| Pregelatinised maize starch | 50 |
| Aspartame | 5 |
| Acesulfame potassium | 5 |
| Magnesium stearate | 3.75 |

### EXAMPLE 3

### Formulation

### Tablet to be Sucked, Having a Weight of 500 Mg

| Constituents | Amount (mg) |
|---|---|
| Agomelatine | 1 |
| Sucrose for direct compression | 435.25 |
| Pregelatinised maize starch | 50 |
| Aspartame | 5 |
| Acesulfame potassium | 5 |
| Magnesium stearate | 3.75 |

### EXAMPLE 4

### Formulation

### Solid Obtained by Infection-Moulding

| Constituents | Amount (mg) |
|---|---|
| Agomelatine | 2 |
| Hydroxypropylcellulose | 333 |
| Mannitol | 150 |
| Aspartame | 7.5 |
| Acesulfame potassium | 7.5 |

The solid is prepared by mixing the constituents. The mixture is then poured into a feed hopper for an Arburg S250 type injection-moulding press. The mixture is heated to 140° C. by the five heating zones of the plasticising unit regulated incrementally from 95° C. to 140° C. and is injected at 1500 bar into a multi-cavity mould.

### EXAMPLE 5

A single-administration pharmacokinetic test was carried out in 12 healthy male volunteers. Three formulations—two orodispersible tablets and one tablet to be sucked—were tested. As the orodispersible pharmaceutical formulations can be administered oromucosally (on top of the tongue) or sublingually, these two modes of administration were also considered and assessed. Accordingly, the 12 volunteers were alternatively given:
an orodispersible tablet having a central core containing 2 mg of agomelatine, administered by sublingual application (central core containing 2 mg of agomelatine, 0.35 mg of magnesium stearate, 67.65 mg of Starlac®; outer layer containing 236.25 mg of Starlac®, 2.5 mg of acesulfame potassium, 2.5 mg of aspartame, 7.5 mg of citric acid and 1.25 mg of magnesium stearate);
a three-layer orodispersible tablet containing 2 mg of agomelatine, administered by sublingual application (central core containing 2 mg of agomelatine, 0.35 mg of magnesium stearate, 67.65 mg of Starlac®; 2 outer layers, each of 125 mg, containing 238.75 mg of Starlac®, 2.5 mg of sucralose, 7.5 mg of citric acid and 1.25 mg of magnesium stearate);
a three-layer orodispersible tablet containing 2 mg of agomelatine, administered by oromucosal application;
a tablet to be sucked, according to the invention, containing 2 mg of agomelatine, administered in the buccal cavity (tablet of Example 1).

Samples of plasma were taken immediately prior to treatment and then regularly from 2 minutes to 24 hours after administration of the tablet, and the following pharmacokinetic parameters were measured: Cₘₐₓ (maximum concentration observed following administration) and AUC (absorption-elimination result for the active ingredient administered). The following results were obtained:

| | | | | Absolute |
|---|---|---|---|---|
| | | Cₘₐₓ | AUC | bioavailability |
| | Formulation | (ng/ml) | (ng · h/ml) | (%) |
| Prior art | Central core, orodispersible, | 8.3 ± 4.6 | 5.7 ± 2.7 | 19% ± 8.6 |
| | 2 mg of agomelatine | (7.8) | (5.4) | |
| | Sublingual administration | | | |
| | Three-layer, orodispersible, | 7.8 ± 3.4 | 5.1 ± 2.1 | 17% ± 6.6 |
| | 2 mg of agomelatine | (7.5) | (5.2) | |
| | Sublingual administration | | | |
| | Three-layer, orodispersible, | 8.7 ± 2.5 | 6.1 ± 1.6 | 20% ± 4.9 |
| | 2 mg of agomelatine | (8.7) | (6.1) | |
| | Oromucosal administration | | | |
| Example 1 | Tablet to be sucked, | 16 ± 4.9 | 11 ± 2.8 | 38% ± 8.9 |
| | 2 mg of agomelatine | (16.0) | (11.0) | |
| | Buccal administration | | | |

The results obtained show that the formulation developed according to the present invention makes it possible to double the pharmacokinetic parameters obtained using the orodispersible formulations previously described. The AUC, in direct correlation to the bioavailability, shows that this new formulation makes it possible to double the bioavailability obtained, compared to the orodispersible tablet.

### EXAMPLE 6

A second single-administration pharmacokinetic test was carried out in 12 healthy male volunteers. Four formulations—two orodispersible tablets and two tablets to be sucked—were tested. The orodispersible pharmaceutical formulations were administered sublingually. Accordingly, the 12 volunteers were alternatively given:
a tablet to be sucked, containing 0.5 mg of agomelatine, administered in the buccal cavity (tablet of Example 2);
a tablet to be sucked, containing 1 mg of agomelatine, administered in the buccal cavity (tablet of Example 3);
an orodispersible tablet having a central core containing 0.5 mg of agomelatine, administered by sublingual application (central core containing 0.5 mg of agomelatine, 0.35 mg of magnesium stearate, 69.15 mg of Starlac®; outer layer containing 236.25 mg of Starlac®, 2.5 mg of acesulfame potassium, 2.5 mg of aspartame, 7.5 mg of citric acid and 1.25 mg of magnesium stearate);
an orodispersible tablet having a central core containing 1 mg of agomelatine, administered by sublingual application (central core containing 1 mg of agomelatine, 0.35 mg of magnesium stearate, 68.65 mg of Starlac®; outer layer containing 236.25 mg of Starlac®, 2.5 mg of acesulfame potassium, 2.5 mg of aspartame, 7.5 mg of citric acid and 1.25 mg of magnesium stearate).

Samples of plasma were taken immediately prior to treatment and then regularly from 2 minutes to 24 hours after administration of the tablet, and the following pharmacokinetic parameters were measured: Cₘₐₓ (maximum concentration observed following administration) and AUC (absorption-elimination result for the active ingredient administered). The relative bioavailability (AUC_{tablet according to the invention}/AUC_{orodispersible tablet from the prior art}) was calculated for each individual and the results obtained were averaged. All the results obtained are set out in the following Table:

| | | | | Relative |
|---|---|---|---|---|
| | | | | bioavailability |
| | | Cₘₐₓ | AUC | (F_{tablet to be sucked}/ |
| | Formulation | (ng/ml) | (ng · h/ml) | F_{orodispersible tablet}) |
| Prior art | Central core, orodispersible, | 3.6 ± 1.5 | 2.7 ± 0.97 | — |
| | 0.5 mg of agomelatine | (3.2) | (2.6) | |
| | Sublingual administration | | | |
| Example 2 | Tablet to be sucked, | 5.3 ± 1.2 | 3.8 ± 0.93 | 1.55 ± 0.65 |
| | 0.5 mg of agomelatine | (5.1) | (4.0) | (1.38) |
| | Buccal administration | | | |
| Prior art | Central core, orodispersible, | 6.1 ± 2.6 | 4.3 ± 1.9 | — |
| | 1 mg of agomelatine | (5.9) | (3.7) | |
| | Sublingual administration | | | |
| Example 3 | Tablet to be sucked, | 9.4 ± 3.0 | 7.3 ± 2.2 | 1.86 ± 0.75 |
| | 1 mg of agomelatine | (9.0) | (7.0) | (1.54) |
| | Buccal administration | | | |

The results obtained show that the formulation developed according to the present invention, at different dosages (0.5 and 1 mg of active ingredient), makes it possible to significantly increase the pharmacokinetic parameters compared to those obtained with the orodispersible formulations described in the prior art at the same dosages, and wherein the active principle is under the same form. Accordingly, the relative bioavailability of the new formulation according to the invention, compared to the orodispersible formulation in the prior art, shows an improvement by a factor ranging from 1.5 to 1.8.

1. A solid buccal controlled release pharmaceutical composition comprising agomelatine or one of its hydrates, complexes, co-crystals, crystalline forms, addition salts with a pharmaceutically acceptable acid or base, and one or more excipients selected from diluents, binders, flow agents, lubricants, coating agents, plasticisers, flavourings, counter-irritants and sweeteners, wherein the composition is intended for systemic action, and wherein the composition has an absolute bioavailability greater than 25%.

2. A solid buccal controlled release pharmaceutical composition comprising agomelatine or one of its hydrates, complexes, co-crystals, crystalline forms, addition salts with a pharmaceutically acceptable acid or base, and one or more excipients selected from diluents, binders, flow agents, lubricants, coating agents, plasticisers, flavourings, counter-irritants and sweeteners, wherein the composition is intended for systemic action, and wherein the composition has a bioavailability greater than that obtained for an orodispersible tablet with an oromucosal absorption containing the active principle agomelatine under the same form and at the same dosage.

3. The pharmaceutical composition according to claim 1, wherein the composition comprises a diluent having a very good compression capability and providing an erodable character.

4. The pharmaceutical composition according to claim 3, wherein the diluent is a polyol or a saccharide.

5. A solid buccal controlled release pharmaceutical composition comprising agomelatine or one of its hydrates, complexes, co-crystals, crystalline forms, addition salts with a pharmaceutically acceptable acid or base, and one or more excipients selected from diluents, binders, flow agents, lubricants, coating agents, plasticisers, flavourings, counter-irritants and sweeteners, wherein the composition is intended for systemic action, and wherein the composition is a tablet to be sucked.

6. The pharmaceutical composition according to claim 1, wherein the composition is a tablet to be sucked.

7. A solid buccal controlled release pharmaceutical composition comprising agomelatine or one of its hydrates, complexes, co-crystals, crystalline forms, addition salts with a pharmaceutically acceptable acid or base, and one or more excipients selected from diluents, binders, flow agents, lubricants, coating agents, plasticisers, flavourings, counter-irritants and sweeteners, wherein the composition is intended for systemic action, and wherein the composition is a lozenge to be sucked.

8. The pharmaceutical composition according to claim 1, wherein the composition is a lozenge to be sucked.

9. A solid buccal controlled release pharmaceutical composition comprising agomelatine or one of its hydrates, complexes, co-crystals, crystalline forms, addition salts with a pharmaceutically acceptable acid or base, and one or more excipients selected from diluents, binders, flow agents, lubricants, coating agents, plasticisers, flavourings, counter-irritants and sweeteners, wherein the composition is intended for systemic action, and wherein the composition is a solid obtained by hot-melt extrusion or injection-moulding.

10. The pharmaceutical composition according to claim 1, wherein the composition is a solid obtained by hot-melt extrusion or injection-moulding.

11. A solid buccal controlled release pharmaceutical composition comprising agomelatine or one of its hydrates, complexes, co-crystals, crystalline forms, addition salts with a pharmaceutically acceptable acid or base, and one or more excipients selected from diluents, binders, flow agents, lubricants, coating agents, plasticisers, flavourings, counter-irritants and sweeteners, wherein the composition is intended for systemic action, and wherein the composition is a pastille to be sucked.

12. The pharmaceutical composition according to claim 1, wherein the composition is a pastille to be sucked.

13. The pharmaceutical composition according to claim 1, wherein the agomelatine is obtained in crystalline form II.

14. The pharmaceutical composition according to claim 1, wherein the composition comprises from 0.1 to 25 mg of agomelatine.

15. The pharmaceutical composition according to claim 1, wherein the composition comprises from 0.1 to 10 mg of agomelatine.

16. The pharmaceutical composition according to claim 1, wherein the composition comprises from 0.1 to 5 mg of agomelatine.

17. The pharmaceutical composition according to claim 1, wherein the composition comprises from 0.1 to 3 mg of agomelatine.

18. The pharmaceutical composition according to claims 1, wherein the composition has an absolute bioavailability greater than 30%.

19. The pharmaceutical composition according to claim 1, wherein the relative bioavailability of the tablet to be sucked compared to an orodispersible tablet with an oromucosal absorption having the same dosage and containing the active principle exactly under the same form is at least 1.5.

20. A method of treating a condition selected from major depression, seasonal affective disorder, generalized anxiety disorder, obsessional compulsive disorder, bipolar disorders, sleep disorders, cardiovascular pathologies, pathologies of the digestive system, insomnia and fatigue due to jet-lag, appetite disorders and obesity, and pathologies associated with deregulation of circadian rhythms in a subject in need thereof, comprising administration of an effective amount of a pharmaceutical composition according to claim 1.
